(19) United States
(12) Patent Application Publication
(10) Pub. No.: US 2026/0086008 A1
(43) Pub. Date: Mar. 26, 2026

(54) Test method for reducing test error in shear strength of root-soil composite

(71) Applicant: TARIM UNIVERSITY, Aksu (CN)

(72) Inventors: Dewei WANG, Aksu (CN); Qian XI, Aksu (CN); Xingguang CHI, Aksu (CN); Mao JI, Aksu (CN); Decheng WANG, Aksu (CN); Xufeng WANG, Aksu (CN)

(21) Appl. No.: 19/401,440

(22) Filed: Nov. 26, 2025

Related U.S. Application Data
(63) Continuation of application No. PCT/CN2025/070681, filed on Jan. 6, 2025.

(30) Foreign Application Priority Data
Apr. 29, 2024 (CN) 2024105276685

(51) Int. Cl. G01N 3/24; G01N 33/00; G01N 33/24

(52) U.S. Cl. CPC G01N 3/24; G01N 33/0098; G01N 33/245; G01N 2203/0025; G01N 2203/0218; G01N 2203/0284

(57) ABSTRACT

The present disclosure discloses a test method for reducing test error in shear strength of a root-soil composite, belonging to the field of agricultural engineering testing methods. The method includes: sampling with a ring knife perpendicular to an actual shear surface to ensure consistency between a direction of the actual shear surface of the root-soil composite and a shear surface direction in a direct shear test; determining a shear strength of soil in each layer within an actual shear depth range by stratifying with a minimum thickness of the ring knife; and planning an annular sampling area and performing sampling in various directions along the annular area, thereby reducing test error in determining shear characteristics of the root-soil composite from three aspects.

### CROSS-REFERENCE TO RELATED APPLICATIONS

This application is a continuation of International Application No. PCT/CN2025/070681, filed on Jan. 6, 2025, which claims priority to Chinese Patent Application No. 202410527668.5, filed on Apr. 29, 2024. All of the aforementioned applications are incorporated herein by reference in their entireties.

### TECHNICAL FIELD

The present disclosure belongs to the technical field of agricultural engineering and testing, and particularly relates to a test method for reducing test error in shear strength of a root-soil composite.

### BACKGROUND

Tillage machinery is an important component of agricultural machinery. The design process of tillage machinery requires determining the shear strength of the soil to be worked on, which serves as basic test data and a design basis for tillage components. The current commonly used method for determining the shear strength of field soil involves sampling with a ring knife perpendicular to the ground surface, followed by measurement with a direct shear apparatus, and the measurement result is taken as the shear strength of the soil. However, because soil and plant roots actually coexist in farmland soil, the soil and plant roots actually exist in a form of a root-soil composite, exhibiting anisotropic characteristics. Furthermore, physical parameter values such as root content and water content of the soil differ across different depth ranges. Therefore, the current test methods for determining the shear mechanics of the farmland root-soil composite face the following three problems: First, whether the actual shear surface between the tillage component and the soil during the working process is consistent with the test shear surface in the direct shear test is not considered. If the direction of the test shear surface of the obtained sample is inconsistent with the direction of the actual shear surface during the tillage process, test error caused by the anisotropy of the root-soil composite will occur. Second, the root content, water content, and other properties differ at different depths in the soil, and the shear characteristics of each layer are different. Sampling without stratification will lead to test error caused by differences in physical properties. Third, farmland soil contains a large number of roots. Under the influence of roots in the soil, the root structure and distribution also vary in different directions within the same soil layer. Sampling in a single direction will introduce test error caused by root distribution. Therefore, there is an urgent need to propose a ring knife sampling and testing method that can reduce the test error in determining the shear strength of the root-soil composite, in order to accurately determine the shear strength of the field root-soil composite.

### SUMMARY

In view of the above-mentioned technical problems, the present disclosure provides a test method for reducing test error in shear strength of a root-soil composite. The method mainly includes: sampling with a ring knife perpendicular to an actual shear surface to ensure consistency between a direction of the actual shear surface of the root-soil composite and a shear surface direction in a direct shear test; determining a shear strength of soil in each layer within an actual shear depth range by stratifying with a minimum thickness of the ring knife; and planning an annular sampling area and performing sampling in various directions along the annular area, thereby reducing test error in determining shear characteristics of the root-soil composite from three aspects.

The objective of the present disclosure is achieved through the following technical solution:

A test method for reducing test error in shear strength of a root-soil composite, including the following steps:
Step 1: determining an actual shear surface where a shear process occurs between a tillage component and soil during a tillage process, measuring a dihedral angle between the actual shear surface and a soil horizontal plane when a tillage depth is reached, and measuring an actual shear depth H imparted to the soil by the tillage component during the tillage process;
Step 2: planning sampling along the actual shear surface from a horizontal ground surface to a tillage depth range by stratifying according to a diameter of a ring knife, based on the actual shear depth H generated in the soil during the shear process of the actual tillage, and obtaining stratified measurement depths;
Step 3: planning one annular sampling area in each layer based on the planned stratified measurement depths;
Step 4: preparing a sampling plane according to the dihedral angle α of the actual shear surface within a same depth range, placing the ring knife perpendicular to the sampling plane, and performing sampling in various directions along the planned annular area;
Step 5: respectively acquiring root-soil composite samples within the annular area taken in each layer, and performing a root direct shear test in a stratified manner to obtain test results; and
Step 6: expressing a shear strength of the soil in each layer in a stratified manner based on the test results.

Further, in Step 2, planning sampling along the actual shear surface from the horizontal ground surface to the tillage depth range by stratifying according to the diameter of the ring knife, and obtaining the stratified measurement depths, includes: a length L of the shear surface is L=H/cos α; and the diameter of the ring knife is D, and a number of layers n is n=(H/cos α)/D, a first layer depth H₁ is H₁=D×sin α, a second layer depth H₂ is H₂=2D×sin α, a third layer depth H₃ is H₃=3D×sin α, . . . , and an nth layer depth Hₙ is Hₙ=nD×sin α, where n=1,2,3 . . . .

Further, in Step 3, planning one annular sampling area in each layer based on the planned stratified measurement depths, includes: when the nth layer depth Hₙ is Hₙ=nD×sin α, where n=1,2,3 . . . , preparing an annular sampling area having a boss shape; wherein a radius R₁ of a top surface circle of the boss, which is an inner circle, of the annular sampling area is R₁=k×D, where k=3, 4, 5, . . . , 10, and a radius R₂ of a bottom surface circle of the boss, which is an outer circle, of the annular sampling area is R₂=k×D+H cos α.

Further, in Step 4, when preparing the sampling plane, starting from the inner circle having the radius R₁ of the annular sampling area, tilting towards the outer circle having the radius R₂ to prepare a ring knife sampling surface, wherein the ring knife sampling surface is an outer surface of the boss, and the sampling plane is a tangent plane of the outer surface of the boss; and placing the ring knife perpendicular to the sampling plane for sampling, so that a dihedral angle between the ring knife sampling surface and the soil horizontal plane is α, and wherein R₂−R₁=H cos α, and a number N of samples taken within the annular sampling area is N=π(R₁+R₂)/D.

Further, in Step 4, during an actual ring knife sampling operation, sampling is performed in various directions along the outer surface of the boss at a corresponding sampling depth, while a central axis of a second ring knife remains perpendicular to the sampling plane.

Further, in Step 5, respectively acquiring the root-soil composite samples within the annular area taken in each layer, and performing the root direct shear test in a stratified manner to obtain the test results, includes: using an average value of shear strengths from direct shear tests of the root-soil composite samples within the annular area of each layer as the test result.

The beneficial effects of the present disclosure are:
1. The present disclosure measures the dihedral angle between the actual shear surface and the soil horizontal plane, and then prepares the sampling plane. The present disclosure performs sampling with the ring knife perpendicular to the actual shear surface, ensuring consistency between the direction of the actual shear surface of the root-soil composite and the shear surface direction in the direct shear test, thereby avoiding error caused by anisotropy of the root-soil composite.
2. The present disclosure performs stratified sampling along the actual shear surface using the ring knife diameter as the stratification interval, determines the shear strength of the soil in each layer within the actual shear depth range, and expresses the test results in a stratified manner, thereby avoiding test error caused by differences in root content, water content, and other properties at different depths.
3. The present disclosure plans one annular sampling area within the same depth range, and performs sampling in various directions along the planned annular area. This offsets deviations among the samples caused by different root distribution directions, thereby avoiding test error caused by root structure and distribution in various directions within the same soil layer.

### BRIEF DESCRIPTION OF THE DRAWINGS

FIG. 1 is a flow chart of a test method for reducing test error in shear strength of a root-soil composite provided by an embodiment of the present disclosure.

FIG. 2 is a schematic diagram of the principles of the test method provided by an embodiment of the present disclosure.

FIG. 3 is a schematic diagram of a sampling surface design and a ring knife sampling method provided by an embodiment of the present disclosure.

FIG. 4 is a top view of the ring knife sampling surface of FIG. 3.

Reference numerals in the drawings: 1 horizontal ground surface, 2 root system, 3 ring knife, 4 first ring knife central axis, 5 first direct shear test shear surface, 6 second direct shear test shear surface, 7 second ring knife central axis, 8 tillage component, 9 first movement direction, 10 tillage component rotation center, 11 second movement direction, 12 actual shear surface, 13 soil, 14 ring knife sampling surface, 15 soil horizontal plane, 16 planned ring knife sampling position.

### DETAILED DESCRIPTION OF THE EMBODIMENTS

The present disclosure is described in detail below with reference to the accompanying drawings and embodiments.

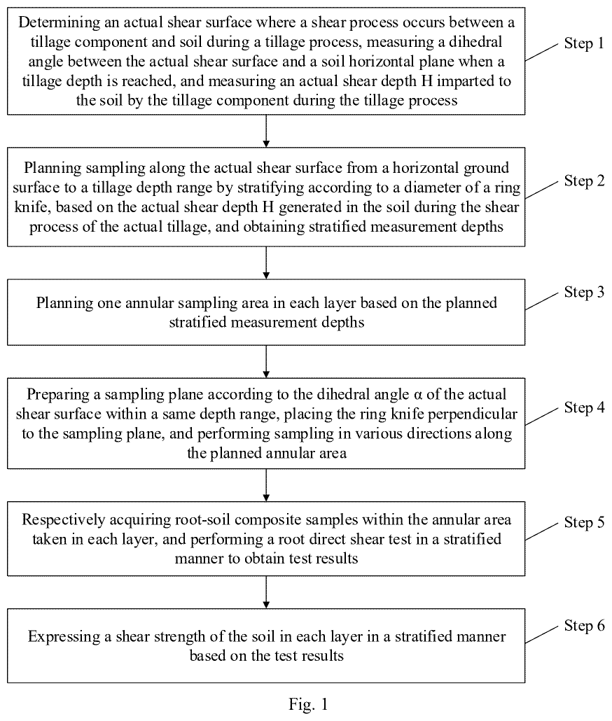

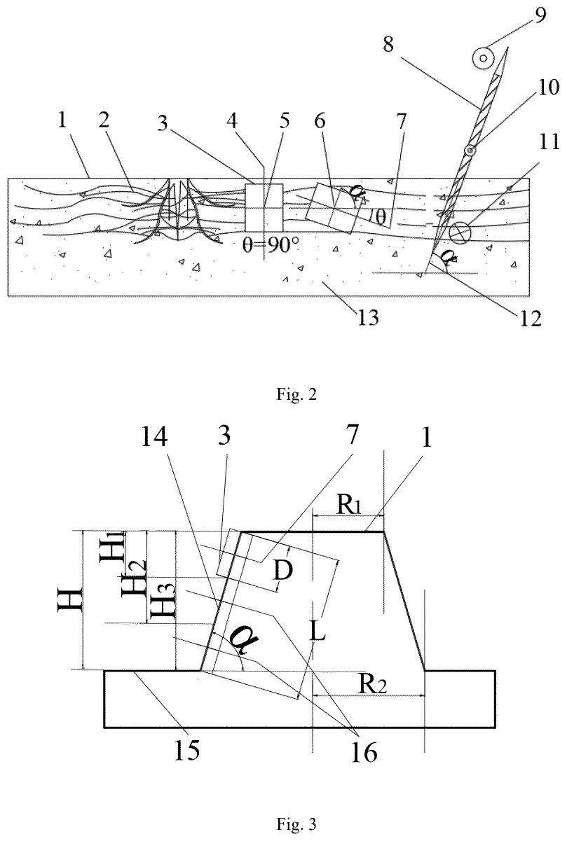

Embodiment: An embodiment of the present disclosure provides a test method for reducing test error in shear strength of a root-soil composite. As shown in FIG. 1, the method includes: determining a dihedral angle of the actual shear surface 12, preparing a sampling plane, stratifying the soil 13 with a height of the ring knife 3, designing an annular sampling area, performing sampling in various directions along the annular sampling area, determining a strength of the root-soil composite at different depths, and expressing test results in a stratified manner. The method can avoid test error caused by inconsistency between the actual shear surface and the shear surface in the direct shear test, test error caused by different physical properties of soil at different depths, and test error caused by different distributions of the root system 2 in the soil. The method specifically includes the following steps:
Step 1: Determining an actual shear surface 12 where a shear process occurs between a tillage component 8 and soil during a tillage process, measuring a dihedral angle between the actual shear surface 12 and a soil horizontal plane 15 when a tillage depth is reached, and measuring an actual shear depth H imparted to the soil by the tillage component 8 during the tillage process.

Wherein, as shown in FIGS. 2-4, determining the actual shear surface 12 where the shear process occurs between the tillage component 8 and the soil during the tillage process refers to determining an angle α formed between the tillage component 8 and the soil horizontal plane 15 during the tillage process; measuring the dihedral angle between the actual shear surface 12 and the soil horizontal plane 15 when the tillage depth is reached refers to measuring the angle α between the actual shear surface 12 formed by tillage and the soil horizontal plane 15; measuring the actual shear depth H imparted to the soil by the tillage component 8 during the tillage process refers to a vertical distance H from the ground surface to the maximum tillage depth when the tillage component enters the soil for operation.
Step 2: Planning sampling along the actual shear surface 12 from a horizontal ground surface 1 to a tillage depth range by stratifying according to a diameter of a ring knife 3, based on the actual shear depth H generated in the soil during the shear process of the actual tillage, and obtaining stratified measurement depths.

Wherein, the actual shear depth H measured in Step 1 is the actual tillage depth range. Specifically, as shown in FIGS. 2-4, planning sampling along the actual shear surface 12 from the horizontal ground surface 1 to the tillage depth range by stratifying according to the diameter of the ring knife 3, and obtaining the stratified measurement depths, includes: a length L of the shear surface is L=H/cos α; the diameter of the ring knife 3 is D, and a number of layers n is n=(H/cos α)/D, a first layer depth H₁ is H₁=D×sin α, a second layer depth H₂ is H₂=2D×sin α, a third layer depth H₃ is H₃=3D×sin α, . . . , and an nth layer Hₙ is Hₙ=nD×sin α, where n=1,2,3 . . . . Wherein, the planned ring knife sampling positions 16, achieved through stratification, are shown in FIG. 3.
Step 3: Planning one annular sampling area in each layer based on the planned stratified measurement depths.

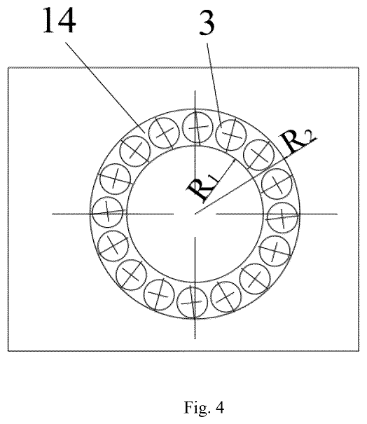

Specifically, when the nth layer depth Hₙ is Hₙ=nD×sin α, where n=1,2,3 . . . , preparing an annular sampling area having a boss shape. Considering practical requirements, the annular sampling area cannot be too large or too small. In this embodiment, as shown in FIGS. 3-4, a radius R₁ of a top surface circle of the boss, which is an inner circle, of the annular sampling area is R₁=k×D, where k=3, 4, 5, . . . 10, and a radius R₂ of a bottom surface circle of the boss, which is an outer circle, of the annular sampling area is R₂=k×D+H cos α.
Step 4: Preparing a sampling plane according to the dihedral angle α of the actual shear surface 12 within a same depth range, and placing the ring knife 3 perpendicular to the sampling plane for sampling, thereby ensuring that the dihedral angle between the sampling plane and the soil horizontal plane 15 is consistent with the dihedral angle between the actual shear surface 12 and the soil horizontal plane 15 in any sampling direction. Simultaneously, a plurality of samples are taken in various directions along the planned annular area. Positive and negative deviations in test results caused by root distribution directions are offset by calculating the average shear strength of the samples, thereby reducing test error.

Specifically, as shown in FIG. 3, when preparing the sampling plane, starting from the inner circle having the radius R₁ of the annular sampling area, tilting towards the outer circle having the radius R₂ to prepare a ring knife sampling surface 14, wherein the ring knife sampling surface 14 is an outer surface of the boss, and the sampling plane is a tangent plane of the outer surface of the boss; and placing the ring knife 3 perpendicular to the sampling plane for sampling, so that a dihedral angle between the ring knife sampling surface 14 and the soil horizontal plane 15 is α, and wherein R₂−R₁=H cos α, and a number N of samples taken within the annular sampling area is N=π(R₁+R₂)/D.

During an actual ring knife sampling operation, sampling is performed in various directions along the outer surface of the boss at a corresponding sampling depth, while a second ring knife central axis 7 remains perpendicular to the sampling plane. This offsets deviations among the samples caused by different root distribution directions, thereby avoiding test error caused by root structure and distribution in various directions within the same soil layer. Concurrently, this sampling method ensures consistency between the direction of the actual shear surface of the root-soil composite and the shear surface direction in the direct shear test, avoiding error caused by anisotropy of the root-soil composite.
Step 5: Respectively acquiring root-soil composite samples within the annular area taken in each layer, and performing a root direct shear test in a stratified manner to obtain test results.

It should be noted that the direct shear test is performed on the acquired samples layer by layer. Because the probability of effects causing overestimation or underestimation is consistent when roots have different distribution directions within the same layer and on the same sampling surface, the average value of the shear strengths from the direct shear tests of the root-soil composite samples within the annular area of each layer can be used as the test result. This can offset test error caused by different root distribution directions in the same layer and same direction of the soil.
Step 6: Expressing a shear strength of the soil in each layer in a stratified manner based on the test results.

It can be understood that performing stratified sampling along the actual shear surface using the ring knife diameter as the stratification interval, determining the shear strength of the soil in each layer within the actual shear depth range, and expressing the test results in a stratified manner can thereby avoid test error caused by differences in root content, water content, and other properties at different depths.

To facilitate a clearer understanding of the present disclosure, the design principles of the present disclosure are specifically elaborated as follows:
I. Overall Design Principles of the Test Method: As shown in FIG. 2, taking an actively penetrating, extremely narrow root-cutting blade with a rotational motion as an example, the root-cutting blade (i.e., the tillage component 8) performs a rotational motion about the tillage component rotation center 10. The root-cutting blade at the upper part of the rotation surface moves outward along the first movement direction 9, while the root-cutting blade at the lower part of the rotation surface moves inward along the second movement direction 11. Under the rotational cutting motion of the root-cutting blade, an actual shear surface 12 forming an angle α with the horizontal direction is created. However, common methods currently used in farmland for determining soil shear characteristics do not consider the shear surface formed during the actual working process between the tillage component and the soil. The method for sampling the root-soil composite is: sampling with a ring knife where the angle θ between its first ring knife central axis 4 and the horizontal ground surface is 90°; and conducting a direct shear test where the first direct shear test shear surface 5 formed during the direct shear test makes an angle α=0° with the horizontal ground surface. Analysis of the schematic diagram in FIG. 2 reveals that when the tillage component 8 operates at an angle α to the horizontal plane, the formed actual shear surface 12 has a dihedral angle α with the horizontal ground surface 1. When α is not zero, the actual shear surface 12 does not coincide with the first direct shear test shear surface 5 formed in the direct shear test. Due to the anisotropic characteristics of the root-soil composite, this leads to a difference between the test result and the actual value, causing test error due to incorrect selection of the sampling surface. Therefore, the method proposed by the present disclosure is preparing a sampling plane that has a dihedral angle α with the soil horizontal plane 15. During sampling, the second ring knife central axis 7 is kept perpendicular to the sampling plane (the angle θ between the second ring knife central axis 7 and the horizontal ground surface 1 is 90°−α). In this case, the second direct shear test shear surface 6 formed during the direct shear test on the sample obtained in this configuration coincides with the actual shear surface 12, thereby overcoming the test error caused by incorrect selection of the sampling surface.
II. Sampling Surface Design and Sampling Method: As shown in FIG. 3, to avoid test error caused by differences in physical properties of soil at different depths, the annular sampling area is planned based collectively on the tillage depth H and the angle α between the actual shear surface 12 and the soil horizontal plane. The front view of the entire sampling area is boss-shaped, and the ring knife sampling surface 14 has a dihedral angle α with the soil horizontal plane 15. The length L of the shear surface is L=H/cos α; the diameter of the ring knife is D, and a number of layers n is n=(H/cos α)/D, the first layer depth H₁ is H₁=D×sin α, the second layer depth H₂ is H₂=2D×sin α, the third layer depth H₃ is H₃=3D×sin α, . . . , and the nth layer depth Hₙ is Hₙ=nD×sin α, where n=1,2,3 . . . . When the nth layer depth Hₙ is Hₙ=nD×sin α, where n=1,2,3 . . . , an annular sampling area having a boss shape is prepared. Considering practical requirements, the annular sampling area cannot be too large or too small. A positive integer k, where k=3, 4, 5, . . . , 10, is introduced to establish a relationship between the radius of the annular sampling area and the diameter D of the ring knife 3. The top surface circle radius of the boss is R₁=k×D, where k=3, 4, 5, . . . 10, and the bottom surface circle radius of the boss is R₂=k×D+H cos α. During preparation, starting from the inner circle of the radius R₁ of the annular sampling area, the ring knife sampling surface 14 is prepared by tilting towards the outer circle of the radius R₂. This sampling surface is the outer surface of the boss. The dihedral angle between the sampling surface and the soil horizontal plane is α, and wherein R₂−R₁=H cos α.

One annular sampling area is planned per layer within the same depth range. Sampling is performed along the planned annular area and along the outer surface of the boss, with the second ring knife central axis maintained perpendicular to the ring knife sampling surface. The number N of samples taken within this annular sampling area is N=π(R₁+R₂)/D. The direct shear test is performed on the acquired samples layer by layer. Because the probability of effects causing overestimation or underestimation is consistent when roots have different distribution directions within the same layer and on the same sampling surface, averaging the measured values of the samples within the annular area of each layer can offset test error caused by different root distribution directions in the same layer and same direction of the soil. Finally, after obtaining the measurement results, the test result values for the shear strength of the root-soil composite are expressed in a stratified manner.

It can be understood that the specific descriptions of the present disclosure above are intended only to illustrate the present disclosure and are not to be construed as limiting the technical solutions described in the embodiments of the present disclosure. Persons of ordinary skill in the art should understand that modifications or equivalent substitutions can still be made to the present disclosure to achieve the same technical effects; provided that operational needs are met, all such modifications or substitutions fall within the protection scope of the present disclosure.

What is claimed is:

1. A test method for reducing test error in shear strength of a root-soil composite, comprising the following steps:
Step 1: determining an actual shear surface where a shear process occurs between a tillage component and soil during a tillage process, measuring a dihedral angle between the actual shear surface and a soil horizontal plane when a tillage depth is reached, and measuring an actual shear depth H imparted to the soil by the tillage component during the tillage process;
Step 2: planning sampling along the actual shear surface from a horizontal ground surface to a tillage depth range by stratifying according to a diameter of a ring knife, based on the actual shear depth H generated in the soil during the shear process of the actual tillage, and obtaining stratified measurement depths;
Step 3: planning one annular sampling area in each layer based on the planned stratified measurement depths;
Step 4: preparing a sampling plane according to the dihedral angle α of the actual shear surface within a same depth range, placing the ring knife perpendicular to the sampling plane, and performing sampling in various directions along the planned annular area;
Step 5: respectively acquiring root-soil composite samples within the annular area taken in each layer, and performing a root direct shear test in a stratified manner to obtain test results; and
Step 6: expressing a shear strength of the soil in each layer in a stratified manner based on the test results.

2. The test method according to claim 1, wherein in Step 2, planning sampling along the actual shear surface from the horizontal ground surface to the tillage depth range by stratifying according to the diameter of the ring knife, and obtaining the stratified measurement depths, comprises: a length L of the shear surface is L=H/cos α; and the diameter of the ring knife is D, and a number of layers n is n=(H/cos α)/D, a first layer depth H₁ is H₁=D×sin α, a second layer depth H₂ is H₂=2D×sin α, a third layer depth H₃ is H₃=3D×sin α, . . . , and an nth layer depth Hₙ is Hₙ=nD×sin α, where n=1,2,3 . . . .

3. The test method according to claim 1, wherein in Step 3, planning one annular sampling area in each layer based on the planned stratified measurement depths, comprises: when the nth layer depth Hₙ is Hₙ=nD×sin α, where n=1,2,3 . . . , preparing an annular sampling area having a boss shape; wherein a radius R₁ of a top surface circle of the boss, which is an inner circle, of the annular sampling area is R₁=k×D, where k=3, 4, 5, . . . , 10, and a radius R₂ of a bottom surface circle of the boss, which is an outer circle, of the annular sampling area is R₂=k×D+H cos α.

4. The test method according to claim 3, wherein in Step 4, when preparing the sampling plane, starting from the inner circle having the radius R₁ of the annular sampling area, tilting towards the outer circle having the radius R₂ to prepare a ring knife sampling surface, wherein the ring knife sampling surface is an outer surface of the boss, and the sampling plane is a tangent plane of the outer surface of the boss; and placing the ring knife perpendicular to the sampling plane for sampling, so that a dihedral angle between the ring knife sampling surface and the soil horizontal plane is α, and wherein R₂−R₁=H cos α, and a number N of samples taken within the annular sampling area is N=π(R₁+R₂)/D.

5. The test method according to claim 3, wherein in Step 4, during an actual ring knife sampling operation, sampling is performed in various directions along the outer surface of the boss at a corresponding sampling depth, while a central axis of a second ring knife remains perpendicular to the sampling plane.

6. The test method according to claim 1, wherein in Step 5, respectively acquiring the root-soil composite samples within the annular area taken in each layer, and performing the root direct shear test in a stratified manner to obtain the test results, comprises: using an average value of shear strengths from direct shear tests of the root-soil composite samples within the annular area of each layer as the test result.
